(19) United States
(12) Patent Application Publication
(10) Pub. No.: US 2024/0325199 A1
(43) Pub. Date: Oct. 3, 2024

(54) BEAM SPLITTING DEVICE, OPHTHALMOLOGICAL LASER THERAPY SYSTEM, METHOD FOR SCANNING A PATIENT'S EYE, AND METHOD FOR SPLITTING

(71) Applicant: Carl Zeiss Meditec AG, Jena (DE)

(72) Inventors: Beate BOEHME, Jena (DE); Mark BISCHOFF, Jena (DE)

(21) Appl. No.: 18/282,768

(22) PCT Filed: Feb. 17, 2022
(86) PCT No.: PCT/EP2022/053970

§ 371 Date: Jun. 10, 2024

(30) Foreign Application Priority Data
Mar. 16, 2021 (DE) 10 2021 106 407.4

(51) Int. Cl. A61F 9/008; A61B 18/20; B23K 26/06; B23K 26/067

(52) U.S. Cl. CPC A61F 9/008; B23K 26/0643; B23K 26/067; A61B 2018/20359; A61B 2018/208; A61F 2009/00897

(57) ABSTRACT

The invention relates to a beam splitting device (10) for generating a plurality of laser output beams (90-93) from one laser input beam (60), wherein: the beam splitting device (10) has a first beam multiplier element (20) for generating two intermediate beams (75, 76) from the laser input beam (60); the first beam multiplier element (20) has a first polarising beam splitter (22, 42), a second polarising beam splitter (24, 44) and at least one first deflection element (26, 46) for deflecting an intermediate beam (76) by a specified angle: the beam splitting device (10) is designed in such a way that, when the laser input beam (60) is irradiated onto the first polarising beam splitter (22, 42) of the first beam multiplier element (20), the laser input beam (60) is split into the first intermediate beam (75) and the second intermediate beam (76) by means of the first polarising beam splitter (22, 42) of the first beam multiplier element (20); the two intermediate beams (75, 76) span the x-y plane, the second intermediate beam (76) is deflected by the first deflection element (26) by a specified angle, in particular approximately 90° or approximately 180°, the first intermediate beam (75) and the second intermediate beam (76) are irradiated onto the second polarising beam splitter (24) of the first beam multiplier element (20) in such a way that the first intermediate beam (75) and the second intermediate beam (76) radiate away from the second polarising beam splitter (24) of the first beam multiplier element (20) at a substantially parallel mutual offset or with a specified angular difference, in particular of less than 3 mrad, preferably less than 1.4 mrad, particularly preferably less than 0.6 mrad.

This application claims priority to German patent application DE 10 2021 106 407.4, filed Mar. 16, 2021, the entire content of which is incorporated herein by reference.

### TECHNICAL FIELD

The invention relates to a beam splitting device, an ophthalmological laser therapy system, a method for scanning a patient's eye and a method for splitting a laser input beam.

### BACKGROUND

In eye surgery or eye measurement, a plurality of laser beams are often moved or scanned simultaneously over the patient's eye. There are a multiplicity of beam splitting devices for creating the plurality of laser beams from one laser beam. Disadvantages of the beam splitting devices known to date include the beam splitting devices being complicated and requiring much space. Moreover, some of the laser beam or some of the intensity/power of the incoming laser beam is lost in beam splitting devices known to date, with the result that the laser input beam must have a very high power.

### SUMMARY OF THE INVENTION

Example embodiments of the invention include a beam splitting device, an ophthalmological laser therapy system and a method for scanning a patient's eye and a method for splitting a laser input beam, which, in technically simple fashion and substantially without power losses, allow the creation of a plurality of laser beams from one laser beam in technically simple fashion and which allow a plurality of laser beams created from one laser beam to be scanned over the patient's eye.

An example embodiment of the invention includes a beam splitting device for creating a plurality of laser output beams from a laser input beam, wherein the beam splitting device comprises a first beam multiplier element for creating two intermediate beams from the laser input beam, wherein the first beam multiplier element comprises a first polarizing beam splitter, a second polarizing beam splitter, and at least one first deflection element for deflecting an intermediate beam through a given angle, wherein the beam splitting device is designed such that, if the laser input beam is radiated at the first polarizing beam splitter in the first beam multiplier element, then the laser input beam is split into the first intermediate beam and the second intermediate beam by use of the first polarizing beam splitter in the first beam multiplier element, with the two intermediate beams spanning the x-y-plane, the second intermediate beam is deflected by the first deflection element through a given angle, for example approx. 90° or approx. 180°, and the first intermediate beam and the second intermediate beam are radiated at the second polarizing beam splitter in the first beam multiplier element, in such a way that the first intermediate beam and the second intermediate beam radiate from the second polarizing beam splitter in the first beam multiplier element in a manner offset substantially in parallel to one another or with a given angular difference, for example of less than 3 mrad (milliradians), in another example less than 1.4 mrad, and in a further example less than 0.6 mrad.

An advantage hereof is that a plurality of laser beams or laser output beams (which may also be referred to as intermediate beams) can be created from one laser beam in technically simple fashion. The laser beams or laser output beams or intermediate beams may run parallel to one another or make an angle with respect to one another. Moreover, the beam splitting device requires only little space. Moreover, substantially no intensity or power of the laser input beam is lost in the beam splitting device. All intermediate beams or component beams created become or contribute to the laser output beams. What is also advantageous in this respect is that the beam splitting device is constructed very easily from a technical point of view and has a compact form. The intermediate beams can be the laser output beams. In the case of a laser input beam at a wavelength of 1 μm and with a beam diameter of 5 mm, the angles of 3 mrad, 1.4 mrad and 0.6 mrad correspond to path differences of 15 wavelengths, 7 wavelengths and 3 wavelengths, respectively (according to tan(angle)=path difference in wavelengths*wavelength/beam diameter).

Another example embodiment of the invention includes an ophthalmological laser therapy system for treating a patient's eye, wherein the laser therapy system comprises a laser generation device for emitting a laser input beam, a beam splitting device for creating two or four laser output beams from the laser input beam, as described hereinabove, and a scanning device for moving the laser output beams over the patient's eye.

What is advantageous in this respect is that a plurality of laser beams or laser output beams which can be moved over the patient's eye can be created from one laser beam in technically simple fashion. The laser beams or laser output beams may run parallel to one another or make an angle with respect to one another. Moreover, the ophthalmological laser therapy system requires only a small amount of space. Additionally, substantially no intensity or power of the laser input beam is lost in the beam splitting device. All intermediate beams or component beams created become or contribute to the laser output beams of the ophthalmological laser therapy system.

Another example embodiment of the invention includes a method for scanning a patient's eye with two or four laser output beams, the method comprising the following steps: radiating a laser input beam into a beam splitting device as a described hereinabove; emitting two or four laser output beams from the beam splitting device; and scanning the laser output beams over at least a portion of the patient's eye.

What is advantageous in this respect is that a plurality of laser beams or laser output beams are created from one laser beam in technically simple fashion and moved over the patient's eye. The laser beams may run parallel to one another or make an angle with respect to one another. The method requires only a small volume or a small amount of space for implementation. Additionally, the intensity or power of the laser output beams is substantially not reduced relative to the laser input beam since all intermediate beams or component beams created contribute to the laser output beams.

A further example embodiment of the invention includes a method for splitting a laser input beam into two laser output beams, the method comprising the following steps: radiating a laser input beam at a first polarizing beam splitter in a first beam multiplier element; splitting the laser input beam into a first intermediate beam and a second intermediate beam by use of the first polarizing beam splitter in the first beam multiplier element; deflecting the second intermediate beam in the first beam multiplier element through a given angle, for example approx. 90° or approx. 180°; and radiating the first intermediate beam and the second intermediate beam at a second polarizing beam splitter in the first beam multiplier element, in such a way that the first intermediate beam and the second intermediate beam emerge from the second polarizing beam splitter in the first beam multiplier element in a manner offset substantially in parallel to one another or with a given angular difference, for example less than 3 mrad, in another example less than 1.4 mrad, and in a further example less than 0.6 mrad.

An advantage hereof is that a plurality of laser output beams (the intermediate beams can be the laser output beams) are created from one laser input beam in technically simple fashion. Only little volume or space is required for the method. Moreover, only little power or intensity of the laser input beam is lost vis-à-vis the laser output beams by virtue of this method.

According to an example embodiment of the beam splitting device, the first polarizing beam splitter in the first beam multiplier element and/or in the second beam multiplier element is aligned vis-à-vis the second polarizing beam splitter in a manner rotated through a given angle, for example approx. 90°, about an axis, which is referred to as the z-axis and which runs perpendicular to the x-y-plane, the distance between the first polarizing beam splitter and the first deflection element being greater than or less than the distance between the second polarizing beam splitter and the first deflection element. An advantage hereof is that, if the deflection element has no tilt, the intermediate beams or the laser output beams run parallel to the laser input beam. Moreover, the intermediate beams can hereby be made to span a plane, in which the laser input beam is located, in a technically simple manner.

According to an example embodiment of the beam splitting device, the first polarizing beam splitter in at least one of the beam multiplier elements is aligned vis-à-vis the second polarizing beam splitter in a manner rotated through an angle not equal to 90°, in the range of approx. 90°±3 mrad/2, for example in the range of approx. 90°±1.4 mrad/2, and in another example in the range of approx. 90°±0.6 mrad/2, about an axis, which is referred to as the z-axis and which runs perpendicular to the x-y-plane. An advantage hereof is that the intermediate beams or laser output beams are caused not to run in parallel in technically simple fashion. The angle between the laser output beams can be adjusted or modified in technically simple fashion as a result of the angle of the two beam splitters with respect to one another.

According to an example embodiment of the beam splitting device, the deflection element comprises a prism, for example a corner cube. An advantage hereof is that a deflection of the beam through 180° is achieved in technically particularly simple fashion. For example, the prism can be a 90° prism (with two reflections). In the case of a corner cube, a tilt of the first polarizing beam splitter and/or of the second polarizing beam splitter, for example about the z-axis, can be used to set the beam angle of the intermediate beams or laser output beams.

According to an example embodiment of the beam splitting device, the first beam multiplier element and/or the second beam multiplier element comprise a respective second deflection element, with the deflection surface of the first deflection element extending substantially parallel to the deflection surface of the second deflection element, with the distance between the first polarizing beam splitter and the first deflection element being greater than or less than the distance between the second polarizing beam splitter and the second deflection element. As a result, two mutually parallel offset intermediate beams or output beams can be created in technically particularly simple fashion.

According to an example embodiment of the beam splitting device, the first beam multiplier element and/or the second beam multiplier element comprise a respective second deflection element, with a first deflection surface of the first deflection element not extending parallel to a second deflection surface of the second deflection element and making an angle with the latter of up to 1.5 mrad, for example up to 0.7 mrad, and in another example up to 0.3 mrad. An advantage hereof is that output beams which do not run parallel to one another but have a small angle in relation to one another can be created in technically particularly simple fashion.

According to an example embodiment of the beam splitting device, the first polarizing beam splitter and the second polarizing beam splitter are arranged adjacent to one another. An advantage hereof is that the beam splitting device can be designed to be more compact or more space-saving. By way of example, the beam splitter element or the beam splitting device may be composed, for example adhesively bonded together, from two (or more) glass prisms. The glass prisms can be 90° prisms. Distances between glass prisms can be designed as glass paths.

According to an example embodiment of the beam splitting device, the polarizations of the first intermediate beam and/or component beam and of the second intermediate beam and/or component beam are each rotated by application of a half-wave plate or two quarter-wave plates before the two intermediate beams or component beams strike the second polarizing beam splitter. As a result, an output beam or the output beams can be emitted parallel to the input beam in technically simple fashion.

According to an example embodiment of the beam splitting device, a retarder is arranged between the first beam multiplier element and the second beam multiplier element. An advantage hereof is that the polarization of the intermediate beams can be altered in technically simple fashion. Hence, the polarization of the laser output beams can be adapted in technically simple fashion.

According to an example embodiment of the beam splitting device, the retarder comprises a half-wave plate or a quarter-wave plate. An advantage hereof is that the beam splitting device is particularly reliable and cost-effective. Moreover, it is possible for example that the two beam multiplier elements are arranged relative to one another in a manner rotated through 90° about the x-axis and the laser input beam has a linear polarization at an angle of 45° in relation to the first plane in order to create a first intermediate beam and a second intermediate beam, with, upstream of the half-wave plate, the first intermediate beam having a linear polarization in a first direction and the second intermediate beam having a linear polarization in a second direction, with the first direction running perpendicular to the second direction, and, downstream of the half-wave plate, the first intermediate beam having a linear polarization of +45° in relation to the first direction and the second intermediate beam having a linear polarization of −45° in relation to the second direction.

According to an example embodiment of the beam splitting device, the deflection surface of the first and/or of the second deflection element in the first beam multiplier element and/or of the first and/or second deflection element in the second beam multiplier element and/or the splitter surface of the second beam splitter in the first beam multiplier element and/or of the second beam splitter in the second beam multiplier element is tilted through a given angle with respect to the x-y-plane about an axis parallel to the x-axis, in such a way that the output beams are not in the x-y-plane. An advantage hereof is that the intermediate beams or the laser output beams experience a tilt in the z-direction or are tilted out of the x-y-plane through the angle Φ. The angle through which the intermediate beams or the laser output beams are tilted out of the x-/y-plane can be set by the tilt angle α of the deflection element or of the beam splitter about the x-axis, with the following formula applying per reflection: Φ=2*α.

According to an example embodiment of the beam splitting device, the beam splitting device is designed such that the distance between the intermediate beams and/or the laser output beams can be kept substantially constant in a plane perpendicular to the x-axis. As a result, the patient's eye can be treated particularly precisely and reliably.

According to an example embodiment of the beam splitting device, the beam splitting device is designed such that the distance of the intermediate beams and/or the laser output beams from one another immediately downstream of the second beam multiplier element is in each case less than 10 times, for example less than four times, the largest diameter of the laser output beams in a plane substantially perpendicular to the x-axis. An advantage hereof is that particularly small areas of the patient's eye are reliably treatable.

According to an example embodiment of the beam splitting device, the laser output beams are not in a straight line in a plane perpendicular to the x-axis. An advantage hereof is that large areas of the patient's eye are treatable within a short period of time. For example, the laser output beams can be arranged in circular, elliptical, hexagonal, rectangular, or square fashion or can be arranged at the corners of any other polygon. For example, it is possible that only two laser output beams of the four laser output beams are located in a common plane in each case.

According to an example embodiment of the ophthalmological laser therapy system, the laser therapy system is designed such that the distances of the laser output beams from one another substantially do not change when moving the laser output beams over the patient's eye. As a result, the patient's eye can be examined and/or treated particularly precisely and reliably.

According to an example embodiment of the method for scanning a patient's eye with two or four laser output beams, the laser output beams are moved over the patient's eye, for example in such a way that the distances of the laser output beams from one another substantially do not change when moving the laser output beams over the patient's eye. An advantage hereof is that the patient's eye is examined and/or treated particularly precisely and reliably.

According to an example embodiment of the method for scanning a patient's eye with two or four laser output beams, the laser output beams are not in a straight line in a plane extending perpendicular to the direction of the laser input beam. An advantage hereof is that large areas of the patient's eye can be treated within a short period of time. For example, the laser output beams can be arranged in circular, elliptical, hexagonal, rectangular, or square fashion or can be arranged at the corners of any other polygon.

According to an example embodiment of the method for splitting a laser input beam, the method further comprises the following steps: radiating the two intermediate beams at a first polarizing beam splitter in a second beam multiplier element; splitting each of the two intermediate beams into a first component beam and a second component beam by use of the first polarizing beam splitter in the second beam multiplier element; deflecting the second component beams in the second beam multiplier element through a given angle, for example approx. 90° or approx. 180°; and radiating the first component beams and the second component beams at a second polarizing beam splitter in the second beam multiplier element, in such a way that four laser output beams emerge from the second polarizing beam splitter in the second beam multiplier element substantially in parallel to one another or with a given angular difference, for example of less than 3 mrad, in another example less than 1.4 mrad, and in a further example less than 0.6 mrad. An advantage hereof is that four output beams are created from one input beam in a small volume by this method.

According to an example embodiment of the method for splitting a laser input beam, the second beam multiplier element is arranged vis-à-vis the first beam multiplier element in a manner rotated through a given angle, for example approx. 45° or approx. 90°, about an axis, which is referred to as the x-axis and runs parallel to the laser input beam. An advantage hereof is that there is no need for 1/n-wave plates or retarders.

According to an example embodiment of the method for splitting a laser input beam, a half-wave plate or a quarter-wave plate is arranged between the first beam multiplier element and the second beam multiplier element. An advantage hereof is that the polarization of the intermediate beams can be altered in technically simple fashion. The component beams tilted with respect to one another are polarized in right-hand circular or left-hand circular fashion by the quarter-wave plate. As a result, the orientation in the second beam multiplier element is irrelevant to the input polarization.

The intermediate beams can be the laser output beams. It is also conceivable that the intermediate beams or the laser output beams in turn are input beams for a further beam multiplier element.

1 mrad corresponds to 0.001 radians (rad).

### BRIEF DESCRIPTION OF THE DRAWINGS

The invention is explained in greater detail below with reference to drawings of exemplary embodiments. In the figures:

FIG. 1 is a schematic side view of a first embodiment of the beam splitting device according to the invention;

FIG. 2 is a schematic perspective view of the beam splitting device from FIG. 1;

FIG. 3 is a schematic side view of a second embodiment of the beam splitting device according to the invention;

FIG. 4 is a schematic perspective view of the beam splitting device from FIG. 3;

FIG. 5 is a schematic perspective view of a third embodiment of the beam splitting device according to the invention;

FIG. 6 is a schematic side view of a third embodiment of a beam multiplier element;

FIG. 7 is a schematic side view of a fourth embodiment of a beam multiplier element;

FIG. 8 is a schematic side view of a fifth embodiment of a beam multiplier element;

FIG. 9 is a schematic side view of a sixth embodiment of a beam multiplier element;

FIG. 10 is a schematic side view of a seventh embodiment of a beam multiplier element;

FIG. 11 is a schematic side view of an eighth embodiment of a beam multiplier element;

FIG. 12 is a schematic side view of a ninth embodiment of a beam multiplier element;

FIG. 13 is a schematic side view of a tenth embodiment of a beam multiplier element;

FIG. 14 is a schematic side view of an eleventh embodiment of a beam multiplier element;

FIG. 15 is a schematic side view of a twelfth embodiment of a beam multiplier element; and

FIG. 16 is a schematic side view of a thirteenth embodiment of a beam multiplier element.

The same reference signs are used in the following description for identical parts and parts having an identical effect.

### DETAILED DESCRIPTION

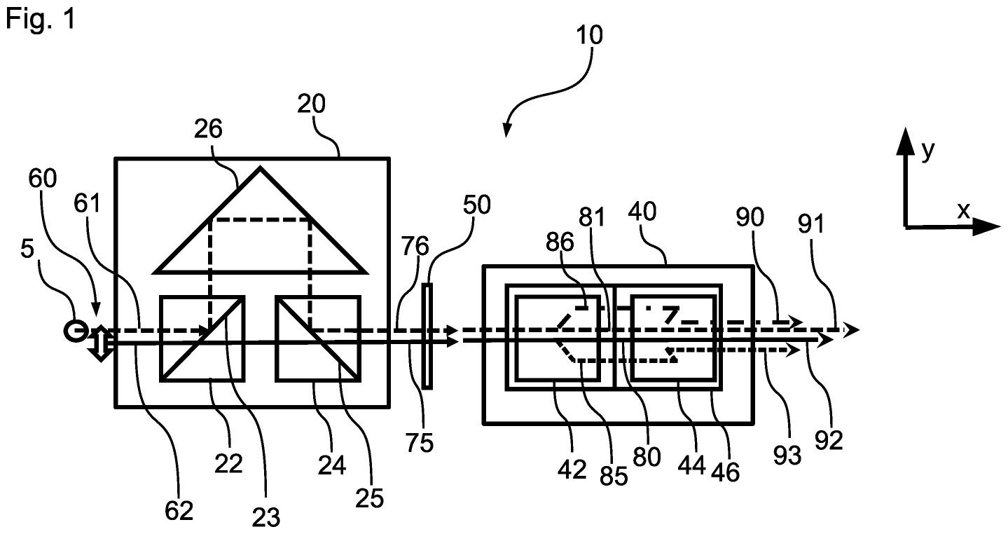

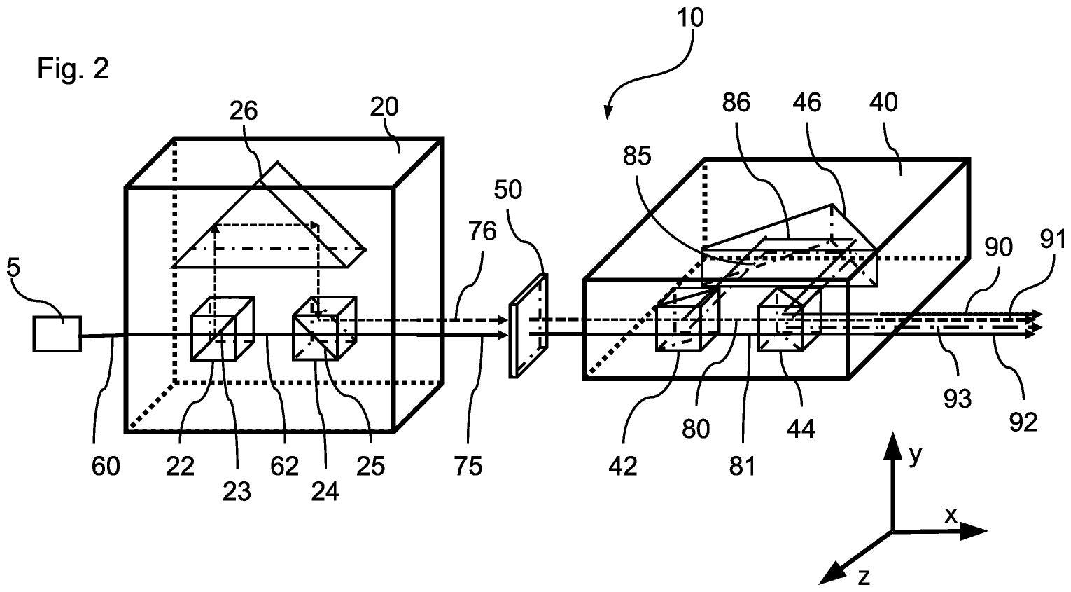

FIG. 1 is a schematic view of a first embodiment of the beam splitting device 10 according to the invention. FIG. 2 shows a schematic perspective view of the beam splitting device 10 from FIG. 1.

The beam splitting device 10 is configured to create two intermediate beams 75, 76 from one laser input beam 60. Four laser output beams 90-93 are created from the two intermediate beams 75, 76 by the beam device, which outputs or emits the said laser output beams.

The beam splitting device 10 comprises a first beam multiplier element 20 and a second beam multiplier element 40. The first beam multiplier element 20 comprises a first polarizing beam splitter 22, a deflection element 26, and a second polarizing beam splitter 24. The second beam multiplier element 40 comprises a first polarizing beam splitter 42, a deflection element 46, and a second polarizing beam splitter 44. Consequently, the first beam multiplier element 20 can have the same structure or design as the second beam multiplier element 40. The first example embodiment of the beam splitting device 10 consequently comprises two instances of a first embodiment of the beam multiplier element.

The coordinate system used in this description uses three mutually perpendicular axes or directions. The x-axis is the axis along which the laser input beam 60 runs (from left to right in FIG. 1). A z-axis runs perpendicular thereto (out of the plane of the drawing in FIG. 1), with the first intermediate beam 75 and the second intermediate beam 76 spanning a plane extending perpendicular to the z-axis downstream of the first polarizing beam splitter 22 in the first beam multiplier element 20. The y-axis (running from bottom to top in FIG. 1) runs perpendicular to the x-axis and perpendicular to the z-axis. Downstream of the first polarizing beam splitter 22 in the first beam multiplier element 20, the second intermediate beam 76 runs in the y-direction or parallel to the y-direction if the laser input beam 60 is radiated at the splitter surface 23 of the first polarizing beam splitter 22 in the first beam multiplier element 20 at an angle of 45°.

The second beam multiplier element 40 in the first beam multiplier element 20 can be arranged vis-à-vis the first beam multiplier element 20 in a manner rotated through 45° or 90° about the x-axis or an axis parallel to the x-axis.

The laser input beam 60 is linearly polarized. By way of example, the laser input beam 60 has a polarization which makes an angle of 45° with respect to the plane of the drawing of FIG. 1.

In FIG. 1, the polarization components of the laser input beam 60 upstream of the first polarizing beam splitter 22 in the first beam multiplier element 20 are represented by two different lines 61, 62. However, only one laser input beam 60 is radiated at the first polarizing beam splitter 22 in the first beam multiplier element 20. The origin of the laser input beam 60 is a laser source 5.

The first polarizing beam splitter 22 in the first beam multiplier element 20, the second polarizing beam splitter 24 in the first beam multiplier element 20, the first polarizing beam splitter 42 in the second beam multiplier element 40, and/or the second polarizing beam splitter 44 in the second beam multiplier element 40 may be, or comprise, a polarizing splitter.

In terms of its direction, a portion of the laser beam or a first component beam or intermediate beam 75 does not change when passing through the first polarizing beam splitter 22 in the first beam multiplier element 20, while the other, second intermediate beam 76 or component beam is deflected through 90°, upward in FIG. 1. Thus, the first polarizing beam splitter 22 in the first beam multiplier element 20 splits the input laser beam 60 into a first intermediate beam 75, which passes through the first polarizing beam splitter 22 unaltered, and a second intermediate beam 76, which is deflected or diverted through 90°. The linear polarization of the first intermediate beam 75 is perpendicular to the linear polarization of the second intermediate beam 76.

The second intermediate beam 76 is steered to a deflection element 26 in the form of a prism. The prism can be a 90° prism, with the second intermediate beam 76 entering through the hypotenuse of the prism. Subsequently, the second intermediate beam 76 is diverted through 90° at a first leg of the prism and subsequently, having traveled over a certain distance, it is diverted or deflected again through 90° at the second leg of the prism, with the result that the second intermediate beam 76 approaches the first intermediate beam 75 again and passes through the hypotenuse of the prism. Now, the second intermediate beam 76 strikes the second polarizing beam splitter 24. However, this typically does not occur at the point where the first intermediate beam 75 passes through the splitter surface 25 of the second polarizing beam splitter 24, but with a slight offset therefrom. The second polarizing beam splitter 24 passes the first intermediate beam 75 coming from the first polarizing beam splitter 22 unaltered, while the second intermediate beam 76 coming from the deflection element 26 is deflected through an angle of 90°.

Now both beams run precisely or substantially in the x-direction. They may run parallel to—but offset from—one another if the deflection element 26 is arranged in a plane perpendicular to the z-axis, which is to say in the plane of the drawing of FIG. 1.

The first polarizing beam splitter 22 may be arranged vis-à-vis the second polarizing beam splitter 24 in a manner rotated through an angle of 90° about the z-axis. This means that the polarizing splitter surfaces or splitter surfaces 23, 25 of the two polarizing beam splitters 22, 24 in the first beam multiplier element 20 are aligned in the x-/y-plane in a manner offset from one another through 90° about the z-axis. As a result, the input beam 60 and the second intermediate beam 76, after the latter has passed through the first polarizing beam splitter 22, the deflection element 26, and the second polarizing beam splitter 24, run parallel to the first intermediate beam 75.

A retarder 50 (waveplate) can be arranged between the first beam multiplier element 20 and the second beam multiplier element 40. The retarder 50 is optional or facultative. The first component beam and the second component beam in the first beam multiplier element 20, also referred to as intermediate beams 75, 76, pass through the retarder 50. The retarder 50 can be what is known as a half-wave plate. The retarder 50 can be arranged and designed such that the polarization direction of the two intermediate beams 75, 76 is rotated through 45°.

Light polarized linearly at 45° with respect to the plane of the drawing of FIG. 1 is radiated at the beam splitting device 10. Immediately or directly upstream of the half-wave plate 50 or immediately/directly downstream of the first beam multiplier element 20, the intermediate beams 75, 76 have a linear polarization in the y-direction/direction of the y-axis and in the z-direction/direction of the z-axis, respectively. Immediately or directly downstream of the half-wave plate 50, the intermediate beams 75, 76 have a linear polarization of +45° with respect to the y-axis and −45° with respect to the z-direction, respectively.

The second beam multiplier element 40 may have structurally the same design as the first beam multiplier element 20. Relative to the first beam multiplier element 20, the second beam multiplier element 40 is rotated through 90° about the x-axis or the direction of the intermediate beams 75, 76. In FIG. 1, the second beam multiplier element 40 is tilted (rotated) backward through 90°. In this case, a retarder 50 is arranged between the first beam multiplier element 20 and the second beam multiplier element 40.

It is also possible that the second beam multiplier element 40 is rotated through 45° about the x-axis vis-à-vis the first beam multiplier element 20. In this case, a retarder 50 cannot be arranged or be present between the first beam multiplier element 20 and the second beam multiplier element 40.

In the case of an angle of 90° between the first beam multiplier element 20 and the second beam multiplier element 40, the intermediate beams 75, 76 in the second beam multiplier element 40 are split into component beams exclusively orthogonal to the plane of the drawing of FIG. 1 or orthogonal to the plane spanned by the intermediate beams 75, 76 in the first beam multiplier element 20. This achieves a particularly efficient adjustment option, as it is decoupled, for the offset of the laser output beams 90-93 from the second beam multiplier element 40 by displacing the deflection element 26 in the first beam multiplier element 20 and the deflection element 46 in the second beam multiplier element 40. The offset between the laser output beams 90-93 from the second beam multiplier element 40 can be varied or set purely by displacing the two deflection elements 26, 46 vis-à-vis the x-/y-plane.

In the second beam multiplier element 40, the intermediate beams 75, 76 which have departed from the first beam multiplier element 20 are incident on a first polarizing beam splitter 42 in the second beam multiplier element 40 which creates two component beams 80, 81, 85, 86 from each intermediate beam 75, 76, which is to say a total of four component beams or intermediate beams, with mutually different polarization alignments.

Two first component beams 80, 81 pass through the first polarizing beam splitter 42 in the second beam multiplier element 40 without changing directions and two second component beams 85, 86 are deflected through 90° by the first polarizing beam splitter 42; into the plane of the drawing in FIG. 1. The deflected second component beams 85, 86 are deflected through 180° or twice through 90° by a deflection element 46. Then, the two deflected second component beams 85, 86 are incident on the second polarizing beam splitter 42 in the second beam multiplier element 40. Here, they are deflected through 90° again. The points at which the second component beams 85, 86 strike the splitter surface 25 of the second polarizing beam splitter 42 typically differ from the points at which the first component beams 80, 81 pass through the splitter surface 25 of the second polarizing beam splitter 44.

Subsequently, the four component beams 80, 81, 85, 86 or the four laser output beams 90-93 can run parallel to—but offset from—one another if the polarizing beam splitters 22, 42 in the first beam multiplier element 20 and in the second beam multiplier element 40 are not tilted vis-à-vis the x-/y-plane or the plane of the drawing of FIG. 1 and the deflection elements 26, 46 are not tilted vis-à-vis the plane of the drawing of FIG. 1.

It is possible that the four laser output beams 90-93 are laser input beams for a further beam multiplier element 20 again, with the result that eight laser output beams are created from four laser input beams 60.

The retarder 50 need not be present. If no retarder 50 is present between the first beam multiplier element 20 and the second beam multiplier element 40, then the second beam multiplier element 40 is rotated through 45° about the x-axis or the direction of the intermediate beams 75, 76 vis-à-vis the first beam multiplier element 20. The two intermediate beams 75, 76 output by the first beam multiplier element 20 are linearly polarized perpendicular to one another. If the deflection element 26 or the prism 26 in the first beam multiplier element 20 is slightly offset, then the intermediate beams 75, 76 have a (small) offset in the x-y-plane. This means that the intermediate beams 75, 76 run parallel to one another in the plane spanned by the x-axis and the y-axis.

After departing from the beam multiplier element 20, the intermediate beams 75, 76 shown in FIG. 1 are in or parallel to the x-y-plane; they have an offset in the y-direction. An offset of the intermediate beams 75, 76 in the z-direction can also be realized by twisting the deflection element 26 about the y-axis.

Additionally or alternatively, it is possible that the splitter surfaces 23, 25 of the first polarizing beam splitter 22 and of the second polarizing beam splitter 24 in the first beam multiplier element 20 are not arranged at an angle of 90° with respect to one another about the z-axis and/or the splitter surfaces of the first polarizing beam splitter 42 and of the second polarizing beam splitter 44 in the second beam multiplier element 40 are not arranged at an angle of 90° with respect to one another about the z-axis. This causes the intermediate beams 75, 76 to not run parallel to one another in the plane of the drawing of FIG. 1.

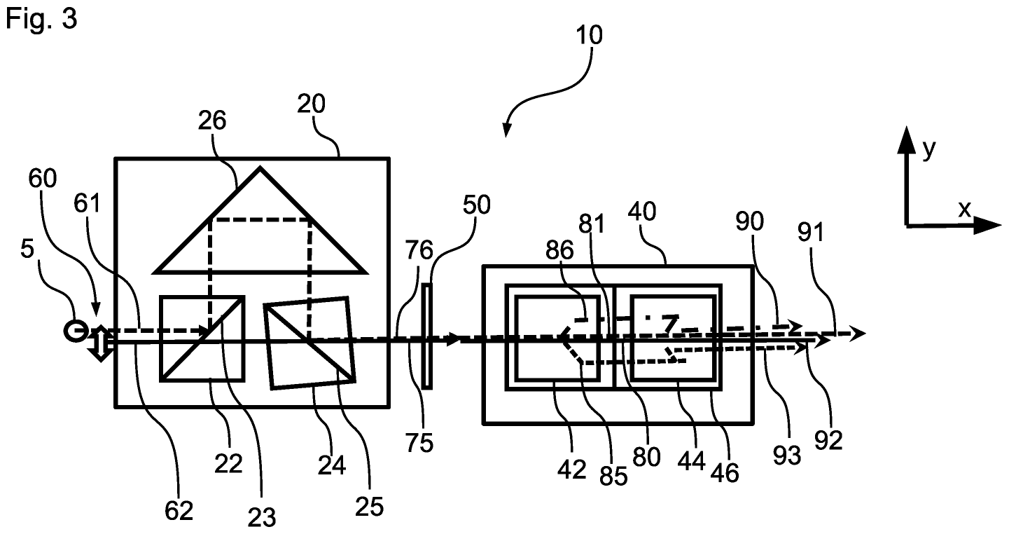

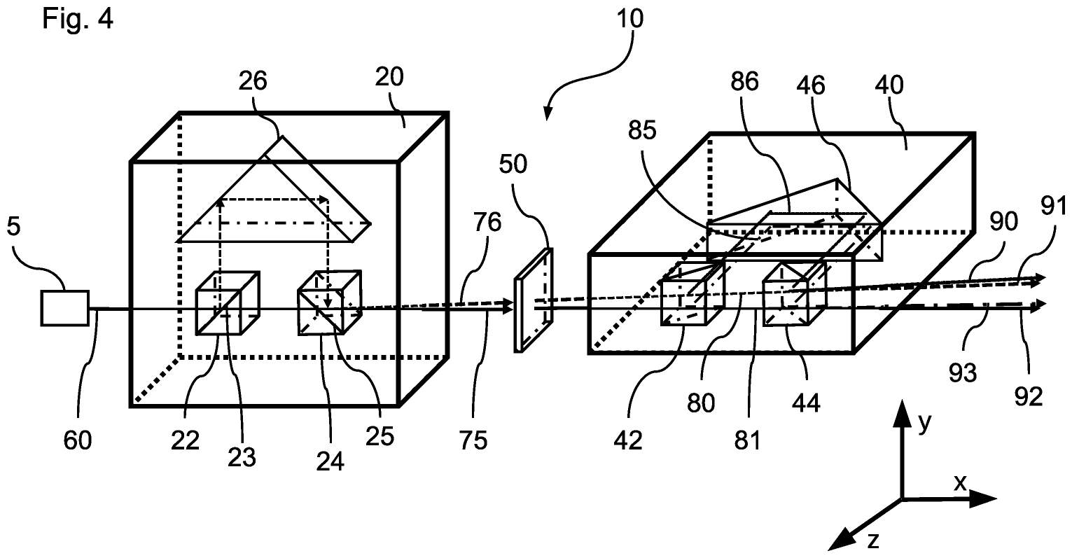

FIG. 3 shows a schematic side view of a second embodiment of the beam splitting device according to the invention. FIG. 4 shows a schematic perspective view of the beam splitting device from FIG. 3.

The second embodiment of the beam splitting device 10 comprises two instances of a second embodiment of the beam multiplier element 20, 40.

In the second embodiment of the beam splitting device 10 or in the second embodiment of the beam multiplier element 20, 40, the splitter surfaces of the first polarizing beam splitter 22 and of the second polarizing beam splitter 24 in the first beam multiplier element 20 are not arranged at an angle of 90° with respect to one another about the z-axis. Moreover, the splitter surfaces of the first polarizing beam splitter 42 and of the second polarizing beam splitter 44 in the second beam multiplier element 40 are not arranged at an angle of 90° with respect to one another about the z-axis. As a result, the intermediate beams and also the output beams respectively do not run parallel to one another in the x-y-plane, which corresponds to the plane of the drawing of FIG. 3 and FIG. 4. The second beam splitter 24, 44 is tilted anticlockwise vis-à-vis the position in FIG. 1 or FIG. 2. In FIG. 3, this tilt of the second beam splitter 24 in the first beam multiplier element is depicted in an exaggerated fashion or not true to scale for illustrative purposes. Usually, the tilt is only approx. 0.3 mrad to approx. 1.5 mrad, or even less than 0.3 mrad, in comparison with a 90° angle relative to one another of the two splitter surfaces of the two beam splitters 22, 24 in the same beam multiplier element. Consequently, the angle with respect to one another of the two splitter surfaces of the two beam splitters 22, 24 in the same beam multiplier element is usually in the range of approx. 90°±1.5 mrad, for example in the range of approx. 90°±0.7 mrad, and in another example in the range of approx. 90°±0.3 mrad, with the value of exactly 90° being excluded from the second embodiment of the beam splitting device. The beams are not emitted in different directions but run parallel to one another or over one another in the case of an angle of exactly 90°.

The angle between the intermediate beams 75, 76 is determined by the tilt angle α of the deflection element 26, 46 about the x-axis or parallel to the x-axis and the angular difference from 90° of the two splitter surfaces 23, 25 of the first polarizing beam splitter 22, 42 and the second polarizing beam splitter 24, 44; in the case of an angle of 90° between the splitter surface 23 of the first polarizing beam splitter 22, 24 and the splitter surface 25 of the second polarizing beam splitter 24, 44, the angle between the intermediate beams 75, 76 is not influenced directly or immediately by the first polarizing beam splitter 22, 42 and the second polarizing beam splitter 24, 44.

When one or more elements of the first beam multiplier element 20 and/or of the second beam multiplier element 40 are tilted vis-à-vis the angular difference of 90° between the splitter surfaces 23, 25 of the two polarizing beam splitters 22, 42, 24, 44 or the x-/y-plane, the four laser output beams 90-93 do not run parallel one another; instead said laser output beams 90-93 are at an angle with respect to one another. In FIG. 3, the laser output beams 90-93 diverge to the right.

The deflection element 26, 46 in the first beam multiplier element 20 and/or in the second beam multiplier element 40 may comprise or be a corner cube. In this case, a deviation of the angle of the two polarizing splitter surfaces or splitter surfaces 23, 25 of the first polarizing beam splitter 22, 42 and of the second polarizing beam splitter 24, 44 about the z-axis or parallel to the z-axis from 90° would lead to a beam angle adjustment or an adjustment of the angle between the intermediate beams 75, 76.

The retarder 50 need not be present. If no retarder 50 is present between the first beam multiplier element 20 and the second beam multiplier element 40, then the second beam multiplier element 40 is rotated through 45° about the x-axis or the direction of the intermediate beams 75, 76 vis-à-vis the first beam multiplier element 20. The two intermediate beams 75, 76 output by the first beam multiplier element 20 are linearly polarized perpendicular to one another. If the deflection element 26 or the prism 26 in the first beam multiplier element 20 is slightly tilted, for example by approx. 0.1 mrad to approx. 0.8 mrad, about the x-axis vis-à-vis the plane of the drawing of FIG. 1, then the intermediate beams 75, 76 have a (small) separation angle Φ in the z-plane. This means that the intermediate beams 75, 76 do not run parallel to one another, but make an angle Φ therebetween, in the plane spanned by the x-axis and the y-axis. The following applies: Φ=2*n*α, where α is the tilt angle of the deflection element 26 about the x-axis or parallel to the x-axis vis-à-vis the plane of the drawing of FIG. 3 and where n is the number of reflections at the deflection element 26.

After departing from the beam multiplier element 20, the intermediate beams 75, 76 shown in FIG. 3 are in or parallel to the x-y-plane and make an angle with respect to one another in this plane. An angular split of the intermediate beams 75, 76 in the x-z-plane can also be realized by twisting the deflection element 26 and/or a polarizing beam splitter 22, 24 about the x-axis.

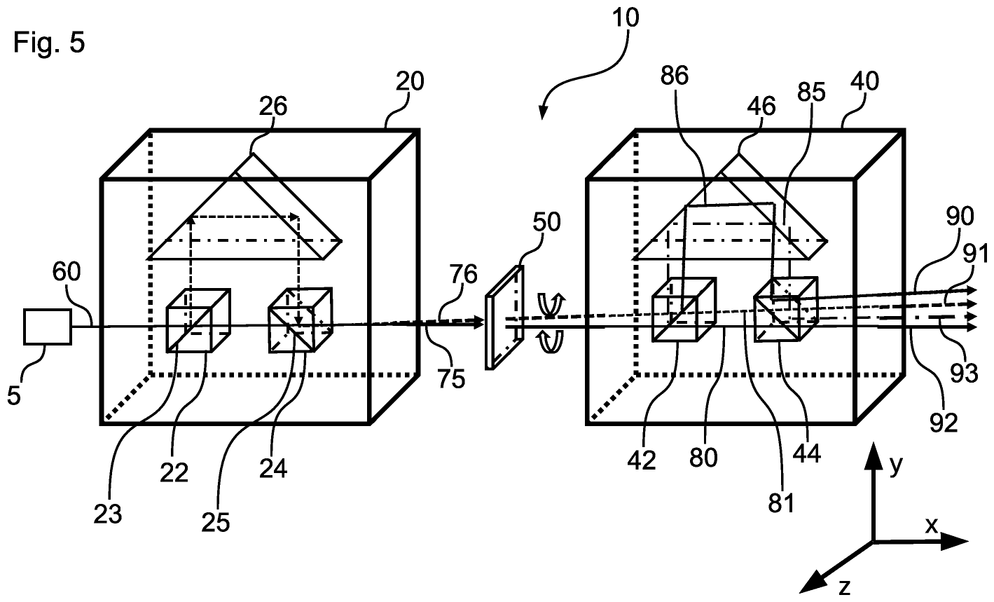

FIG. 5 shows a schematic perspective view of a third embodiment of the beam splitting device 10 according to the invention. The third embodiment of the beam splitting device 10 comprises two instances of a second embodiment of the beam multiplier element 20, 40.

The third embodiment of the beam splitting device 10 differs from the second embodiment of the beam splitting device 10 in that a quarter-wave plate is arranged between the first beam multiplier element 20 and the second beam multiplier element 40, and in that the two beam multiplier elements 20, 40 are not tilted with respect to one another about the x-axis. The component beams tilted with respect to one another are polarized in right-hand circular or left-hand circular fashion by the quarter-wave plate 50. As a result, the orientation in the second beam multiplier element 40 is irrelevant to the input polarization. Consequently, the orientation or alignment of the second beam multiplier element 40 relative to the first beam multiplier element 20 only determines the geometry of the four output beams in relation to one another.

The tilt (i.e., deviation from an angle of exactly 90° with respect to one another about the z-axis) of the splitter surface of the respective second beam splitter 24 relative to the splitter surface of the respective first beam splitter 22 differs in each case. By way of example, the tilt of the splitter surface of the first beam splitter 22 relative to the splitter surface of the second beam splitter 24 is 2 mrad in the first beam multiplier element and the tilt of the splitter surface of the first beam splitter 42 relative to the second beam splitter 44 is 1 mrad in the second beam multiplier element. This means that the angle of the splitter surfaces with respect to one another is for example 90°+1 mrad in the first beam multiplier element while the angle between the splitter surfaces is for example 90°+0.5 mrad in the second beam multiplier element. Consequently, the tilt in the second beam multiplier element may only be half as large as in the first beam multiplier element. Here, too, the tilt angles are depicted in exaggerated fashion or not true to scale for illustrative purposes.

It is also conceivable that the two beam multiplier elements 20, 40 in the third embodiment are designed like in the first embodiment. As a result, the component beams offset parallel to one another are polarized in right-hand circular and left-hand circular fashion by the quarter-wave plate. The intermediate beams 75, 76 tilted with respect to one another can be polarized in right-hand circular and left-hand circular fashion by the quarter-wave plate. As a result, the orientation in the second beam multiplier element 40 is irrelevant to the input polarization.

The position of the output beams 90-93 relative to one another can be altered by rotating the second beam multiplier element 40 about the x-axis.

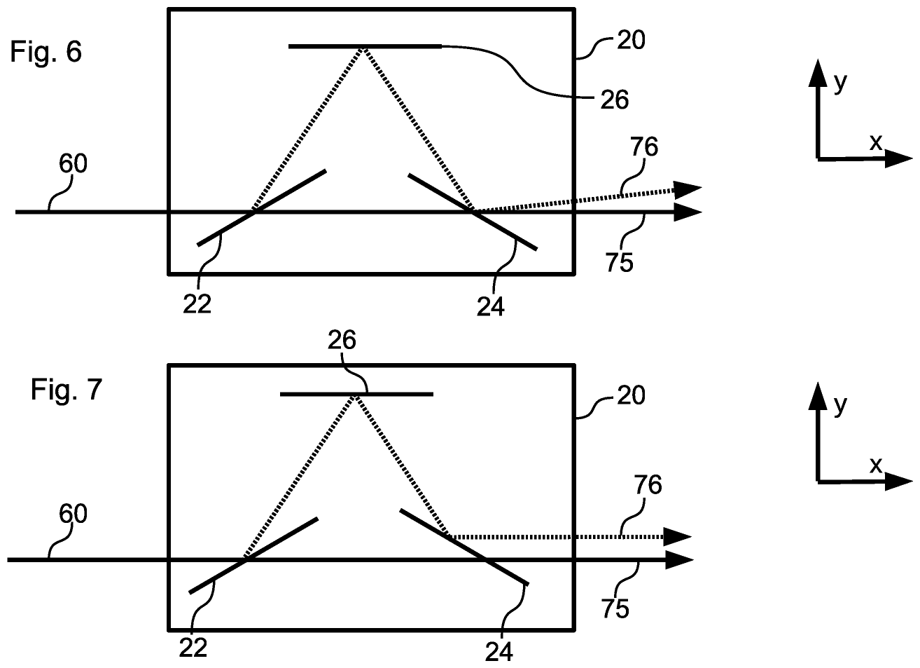

FIG. 6 shows a schematic side view of a third embodiment of a beam multiplier element 20. The first, second, or third embodiment of the beam splitting device 100 may comprise the third embodiment of a beam multiplier element as an alternative or in addition to the first or second embodiment of a beam multiplier element.

In the third embodiment of the beam multiplier element 20, the polarizing beam splitters 22, 24 are not polarization splitter cubes but polarization splitter plates in each case. The two polarizing beam splitters 22, 24 or the splitter surfaces are at an angle with respect to one another, not equal to 90°, in the range of approx. 90°±1.5 mrad, for example in the range of approx. 90°±0.7 mrad, and in the range of approx. 90°±0.3 mrad. As a result, two intermediate beams 75, 76 (which can also be laser output beams) which are emitted by the second splitter surface in different directions are created.

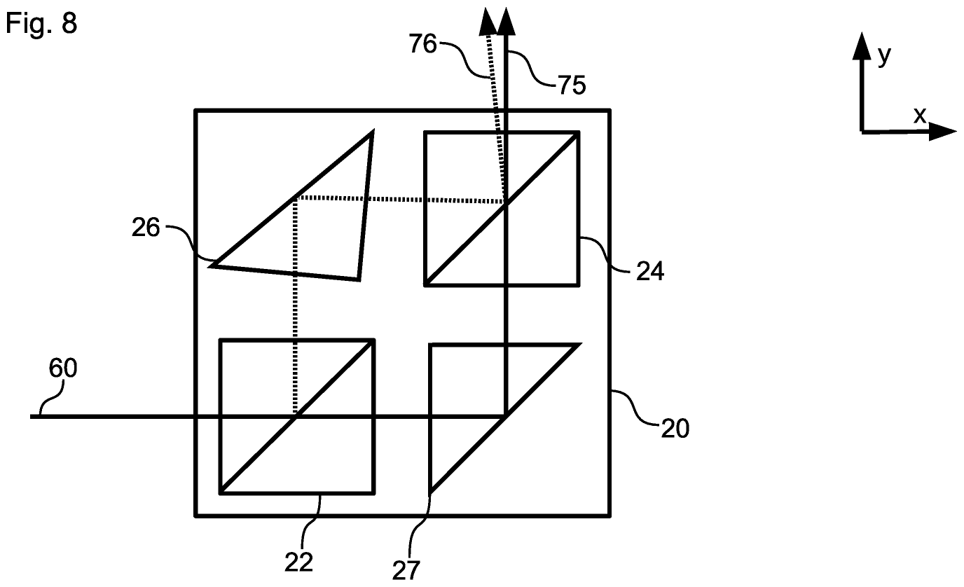

FIG. 7 shows a schematic side view of a fourth embodiment of a beam multiplier element 20. The first or second or third embodiment of the beam splitting device 10 may comprise the fourth embodiment of a beam multiplier element as an alternative or in addition to the first, second, and third embodiment of a beam multiplier element.

In contrast with the third embodiment of the beam multiplier element, the splitter surfaces are at an angle of 90° with respect to one another in the fourth embodiment. However, the splitter surfaces are arranged offset from one another, which is to say the distance in the y-direction between the first splitter surface of the first polarizing beam splitter 22 and the deflection element 26 is greater than or less than the distance in the y-direction between the second splitter surface of the second polarizing beam splitter 24 and the deflection element 26. As a result, the two intermediate beams 75, 76 (which can also be laser output beams) are offset parallel to one another.

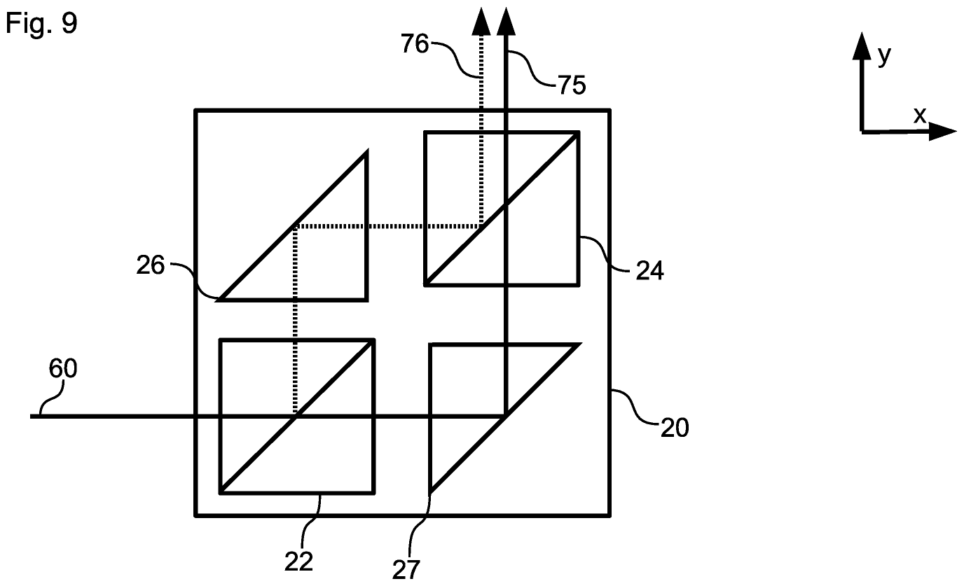

FIG. 8 shows a schematic side view of a fifth embodiment of a beam multiplier element 20. The first or second or third embodiment of the beam splitting device 10 may comprise the fifth embodiment of a beam multiplier element as an alternative or in addition to the first, second, third, and fourth embodiment of a beam multiplier element.

The beam multiplier element 20 comprises two deflection elements 26, 27. The two diffraction elements 26, 27 can each be a prism which is triangular in the side view. A first intermediate beam 75 downstream of the first polarizing beam splitter 22 runs in the direction of the input beam 60. A second intermediate beam 76 downstream of the first polarizing beam splitter 22 runs at an angle of 90° with respect to the first intermediate beam 75. After the respective intermediate beam 75, 76 was deflected through 90° by the deflection element 26, 27, the two intermediate beams 75, 76 are incident on the second polarizing beam splitter 24. Subsequently, the first intermediate beam 75 departs from the beam multiplier element 20 at an angle of 90° with respect to the input beam 60. The second intermediate beam 76 departs from the beam multiplier element 20 at a small angle, which is to say less than for example 3 mrad, for example less than 1.4 mrad, and in another example less than 0.6 mrad, with respect to the first intermediate beam 75. The first deflection element 26 is tilted about the z-axis vis-à-vis the second deflection element 27. The two deflection surfaces of the two diffraction elements 26, 27 do not extend parallel to one another but are at an angle with respect to one another in the range of approx. ±1.5 mrad, for example in the range of approx. ±0.7 mrad, and in another example in the range of approx. ±0.3 mrad.

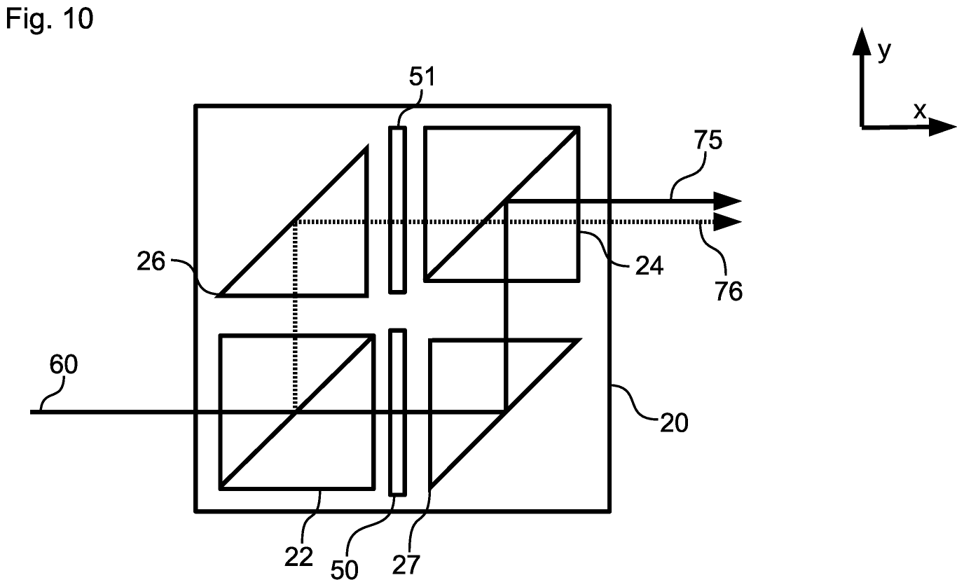

FIG. 9 shows a schematic side view of a sixth embodiment of a beam multiplier element 20. The first or second or third embodiment of the beam splitting device 10 may comprise the sixth embodiment of a beam multiplier element as an alternative or in addition to the first, second, third, fourth and fifth embodiment of a beam multiplier element.

The sixth embodiment of the beam multiplier element 20 differs from the fifth embodiment of the beam multiplier element in that the two deflection surfaces of the two deflection elements 26, 27 extend parallel to one another. However, the deflection surfaces of the two deflection elements 26, 27 or the two deflection elements 26, 27 are arranged offset from one another, which is to say the distance in the y-direction between the first splitter surface of the first polarizing beam splitter 22 and the first deflection element 26 is greater than or less than the distance in the x-direction between the second splitter surface of the second polarizing beam splitter 24 and the deflection element 27. As a result, the two intermediate beams 75, 76 (which can be laser output beams) are offset parallel to one another.

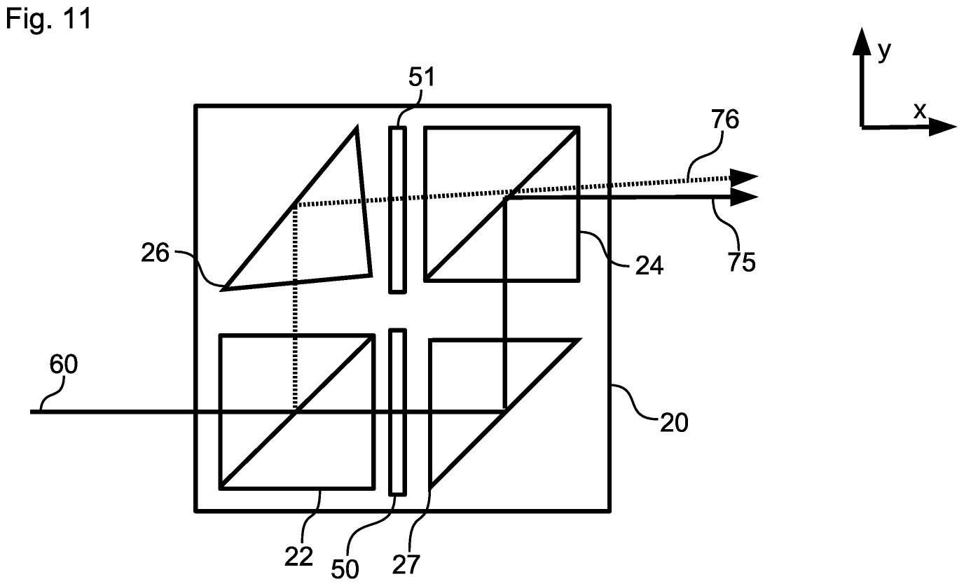

FIG. 10 shows a schematic side view of a seventh embodiment of a beam multiplier element 10. The first or second or third embodiment of the beam splitting device may comprise the seventh embodiment of a beam multiplier element as an alternative or in addition to the first, second, third, fourth, fifth, and sixth embodiment of a beam multiplier element.

The seventh embodiment has a similar design to the fifth embodiment, the difference consisting in the fact that a half-wave plate is arranged between the first deflection element 26 and the second polarizing beam splitter 24 and in that a half-wave plate is arranged between the first polarizing beam splitter 22 and the second deflection element 27. As a result, the second intermediate beam 76, which was deflected through 90° by the first polarizing beam splitter 22, passes through the second polarizing beam splitter 24 unaltered while the first intermediate beam 75 is deflected through 90° by the second polarizing beam splitter 24. Subsequently, the first intermediate beam 75 departs from the beam multiplier element 20 parallel to the input beam 60.

The two deflection surfaces extend parallel to one another. However, the deflection surfaces of the two deflection elements 26, 27 are arranged offset from one another, which is to say the distance in the y-direction between the first splitter surface of the first polarizing beam splitter 22 and the deflection element 26 is greater than or less than the distance in the x-direction between the second splitter surface of the second polarizing beam splitter 22 and the deflection element 27. As a result, the two intermediate beams 75, 76 are offset parallel to one another.

It is also conceivable that the half-wave plates are each arranged so that a half-wave plate is between the second deflection element 27 and the second polarizing beam splitter 24 and a half-wave plate is arranged between the first polarizing beam splitter 22 and the first deflection element 26.

FIG. 11 shows a schematic side view of an eighth embodiment of a beam multiplier element 20. The first or second or third embodiment of the beam splitting device may comprise the eighth embodiment of a beam multiplier element as an alternative or in addition to the first, second, third, fourth, fifth, sixth, and seventh embodiment of a beam multiplier element.

The eighth embodiment has a similar design to the sixth embodiment, the difference consisting in the fact that a half-wave plate is arranged between the first deflection element 26 and the second polarizing beam splitter 24 and in that a half-wave plate is arranged between the first polarizing beam splitter 22 and the second deflection element 27. As a result, the second intermediate beam 76, which was deflected through 90° by the first polarizing beam splitter 22, passes through the second polarizing beam splitter 24 unaltered while the first intermediate beam 75 is deflected through 90° by the second polarizing beam splitter 24. Subsequently, both intermediate beams 75, 76 depart from the beam multiplier element 20 parallel to the input beam 60 and parallel to one another.

The first deflection element 26 is tilted about the z-axis vis-à-vis the second deflection element 27. The two deflection surfaces of the two diffraction elements 26, 27 do not extend parallel to one another but are at an angle with respect to one another in the range of approx. ±1.5 mrad, for example in the range of approx. ±0.7 mrad, and preferably in the range of approx. ±0.3 mrad.

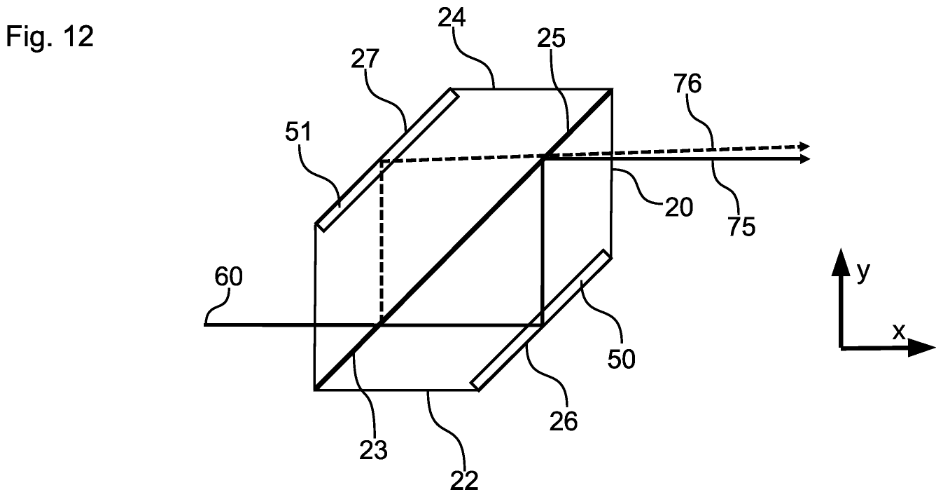

FIG. 12 shows a schematic side view of a ninth embodiment of a beam multiplier element 20. The first or second or third embodiment of the beam splitting device 10 may comprise the ninth embodiment of a beam multiplier element as an alternative or in addition to the first, second, third, fourth, fifth, sixth, seventh, and eighth embodiment of a beam multiplier element. The beam multiplier element 20 comprises two glass prisms, which are arranged, for example in adhesively bonded manner or the like, with their splitter surfaces 23, 25 adjacent to one another. One of the two deflection surfaces or mirror surfaces extends parallel to the splitter surface 23, 25 or to the splitter surfaces 23, 25. The other deflection surface or mirror surface of the deflection elements 26, 27 is aligned with respect to the splitter surface 23, 25 or the splitter surfaces 23, 25 in a manner tilted about the z-axis. For example, the tilt can be approx. 1.5 mrad, for example approx. 0.7 mrad, and in another example 0.3 mrad or less.

The two polarizing beam splitters 22, 24 of the beam multiplier element 20 are arranged adjacently to one another. Consequently, the splitter surfaces 23, 25 of the two polarizing beam splitters 22, 24 extend parallel to one another.

The deflection surfaces comprise a quarter-wave plate or a respective quarter-wave plate is arranged on the deflection surfaces, with the respective intermediate beam 75, 76 passing twice through the respective quarter-wave plate during the deflection or reflection through 90°. This results in the effect of a half-wave plate in each case. This brings about a rotation through 90° of the polarization of the respective intermediate beam 75, 76 during the deflection. The polarization splitter surface is consequently passed respectively once in transmission and once in reflection by the two intermediate beams 75, 76.

The first intermediate beam 75 departs from the beam multiplier element 20 parallel to the input beam 60.

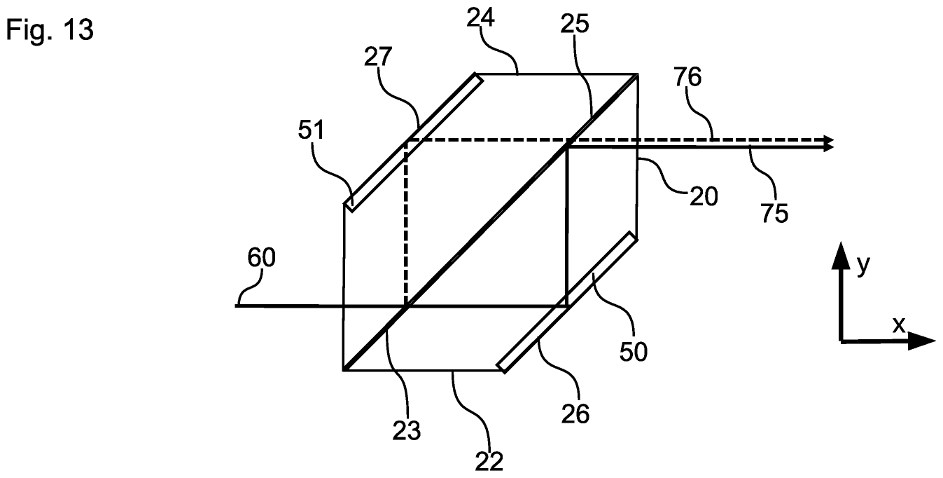

FIG. 13 shows a schematic side view of a tenth embodiment of a beam multiplier element 20. The first or second or third embodiment of the beam splitting device may comprise the tenth embodiment of a beam multiplier element as an alternative or in addition to the first, second, third, fourth, fifth, sixth, seventh, eighth, and ninth embodiment of a beam multiplier element.

The tenth embodiment differs from the ninth embodiment in that the deflection surfaces are aligned parallel to one another and parallel to the splitter surface 23 or the splitter surfaces 23, 25, but the distances between the deflection surfaces and the splitter surfaces 23, 25 are not of the same size. This means that the distance of the first splitter surface 23 of the first polarizing beam splitter 22 from the first deflection surface of the first deflection element 26 is greater than or less than the distance of the splitter surface 25 of the second polarizing beam splitter 24 from the second deflection surface of the second deflection element 27. Consequently, the intermediate beams 75, 76 are emitted by the beam multiplier element 20 offset parallel to one another and parallel to the input beam 60.

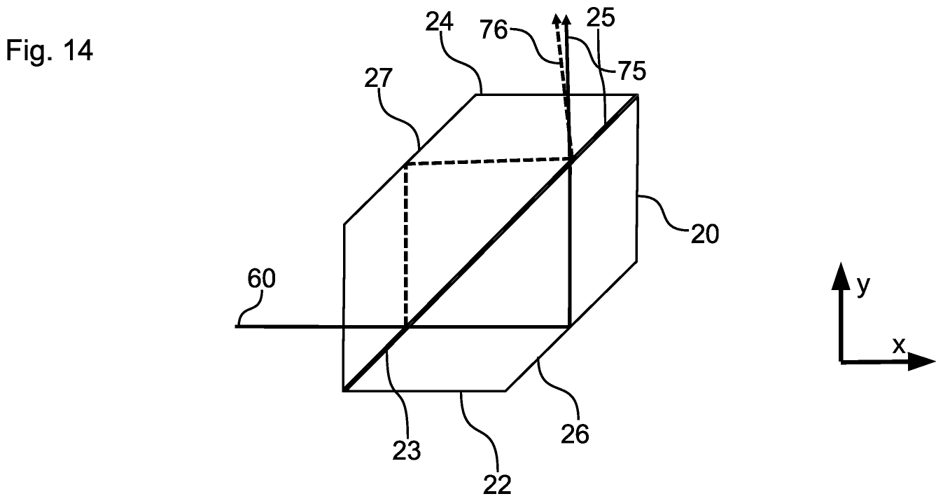

FIG. 14 shows a schematic side view of an eleventh embodiment of a beam multiplier element 20. The first or second or third embodiment of the beam splitting device may comprise the eleventh embodiment of a beam multiplier element as an alternative or in addition to the first, second, third, fourth, fifth, sixth, seventh, eighth, ninth, or tenth embodiment of a beam multiplier element.

The eleventh embodiment differs from the ninth embodiment in that no half-wave plates are present. Consequently, the polarization of the two component beams is not altered. Consequently, one intermediate beam 75 departs from the beam multiplier element at an angle of 90° with respect to the input beam 60. The other intermediate beam 76 is at a small angle, for example less than 3 mrad or less than 1.4 mrad or less than 0.3 mrad, with respect to the other intermediate beam 75.

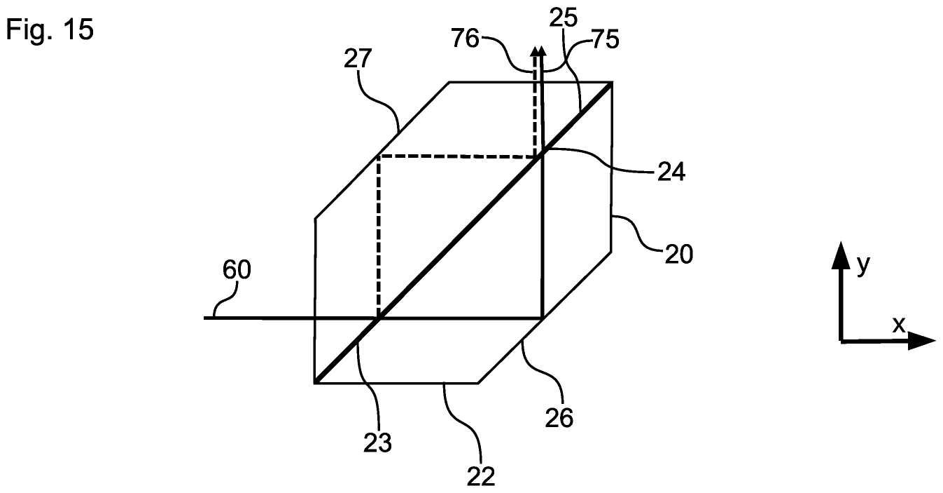

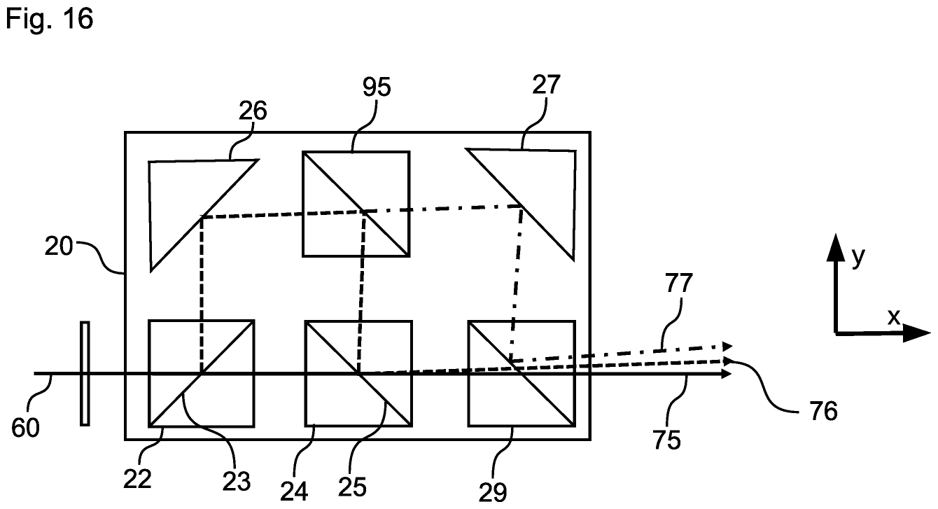

FIG. 15 shows a schematic side view of a twelfth embodiment of a beam multiplier element 20. The first or second or third embodiment of the beam splitting device 10 may comprise the twelfth embodiment of a beam multiplier element as an alternative or in addition to the first, second, third, fourth, fifth, sixth, seventh, eighth, ninth, tenth, or eleventh embodiment of a beam multiplier element.

The twelfth embodiment differs from the tenth embodiment in that no half-wave plates are present. Consequently, the polarization of the two component beams is not altered. Consequently, the two intermediate beams 75, 76 each depart from the beam multiplier element 20 at an angle of 90° with respect to the input beam 60.

FIG. 16 shows a schematic side view of a thirteenth embodiment of a beam multiplier element 20. The first or second or third embodiment of the beam splitting device 10 may comprise the thirteenth embodiment of a beam multiplier element as an alternative or in addition to the first, second, third, fourth, fifth, sixth, seventh, eighth, ninth, tenth, eleventh, or twelfth embodiment of a beam multiplier element.

The thirteenth embodiment of a beam multiplier element 20 creates three intermediate beams from one input beam 60. The first polarizing beam splitter 22 creates two intermediate beams 75, 76. The intermediate beam 76 is deflected through more or less 180° at the deflection element 26 and at the splitter surface of a 50:50 beam splitter 95, and is overlaid on the intermediate beam 75 (at an angle of up to 3 mrad, for example up to 1.4 mrad, in another example up to 0.6 mrad) as described in relation to the second embodiment. Like in the second embodiment, two intermediate beams 75 and 76 are present downstream of the second polarizing beam splitter 24. In the thirteenth embodiment, a third intermediate beam 77 (plotted as the line of dashes and dots) is split off the intermediate beam 76 at the 50:50 beam splitter 95. This third intermediate beam 77 is deflected at a second deflection element 27 and overlaid on the two intermediate beams 75 and 76 at a third polarizing beam splitter 29. The angle of the third intermediate beam 77 in relation to the first component beam 75 and second component beam 76 can be adjusted by tilting the second deflection element 27.

The splitting ratio between the intermediate beams can be set at the first polarizing beam splitter 22 by rotating a retarder (half-wave plate) disposed upstream of the beam multiplier element 20.

Consequently, a substantially loss-free creation of two or four laser output beams 90-93 from one laser input beam 60 is achieved by way of the beam splitting device 10. None of the component beams or intermediate beams are lost or do not contribute to the laser output beams 90-93.

The beam splitting device 10 may be arranged between the laser source and the scanning device. This is possible, for example, if the intermediate beams 75, 76 or the output beams are at different angles to one another.

It is also possible that the beam splitting device 10 is arranged in, or in the vicinity of, an intermediate image plane of the ophthalmological laser therapy system. This applies, for example, if the intermediate beams 75, 76 or the output beams run offset to one another and parallel to one another.

It is also possible that the beam splitting device 10 is arranged in, or in the vicinity of, a pupil plane of the ophthalmological laser therapy system. This applies, for example, if the intermediate beams 75, 76 or the output beams make an angle not equal to zero with respect to one another.

If one of the intermediate beams 75, 76 (in the case of one beam multiplier element) or one of the output beams 90-93 (in the case of two beam multiplier elements) runs in the same direction as the input beam 60, then a single laser beam can be directed at the eye to be treated when the beam splitting device 10 is removed from the beam path. The beam splitting device 10 can be removed from the beam path and can be reintroduced therein. By way of example, this can be implemented by a back-and-forth movement and/or a back-and-forth pivoting of the beam splitting device 10.

For example, the laser beams or output beams or intermediate beams 75, 76 have a spacing from one another of a few airy on the eye to be treated. One airy is or corresponds to the diameter of the (innermost) Airy disk or the (innermost) diffraction disk of the respective laser beam. It is possible that the laser beams or output beams or intermediate beams 75, 76 intersect on the eye to be treated. As a result, the power per laser beam can be lower. The spacing of the centers of the laser beams on the eye to be treated may range between approximately 0.5 airy and 10 airy.

It is conceivable that the beam splitting device 10 comprises one beam multiplier element, two beam multiplier elements or more than two beam multiplier elements, for example three, four, five or more than five beam multiplier elements, which are respectively arranged successively in series.

For example, the active surfaces or splitter surfaces 23, 25 of the polarizing beam splitters 22, 24 (beam splitter surface), of the first deflection element 26 (first deflection surface), of the second deflection element 27 (second deflection surface), and/or of the retarder 50 or retarders (the surfaces thereof) have surface normals which are not parallel to the respectively incident laser beam or the respectively incident laser beams. This avoids reflections. By way of example, the splitters may have a wedge in order to achieve this. The retarder 50 or retarders 50, 51 may each be arranged (slightly) obliquely in the beam path.

The splitter surfaces 23, 25 may also be arranged within a common prism main body or may be embodied as splitter plates. Further, both splitter surfaces and deflection elements may be arranged in a common prism.

On the eye to be treated, the light beams or laser beams may have a spiral shape or be arranged in spiral fashion. Other shapes, for example a line or along a straight line, in a quadrilateral, a hexagonal shape or the like, are conceivable. By way of example, a line would be advantageous if the eye is swept over with the laser beams or output beams in meandering fashion by way of the scanning device.

The polarization of the intermediate beams 75, 76 or output beams, for example by way of retarders 50, 51 and/or an arrangement of a plurality of beam multiplier elements at a given angle about the x-axis, may be such that all laser beams have substantially the same magnitude of intensity on the eye. The differences in the respective intensities may be in the range of up to 20%.

The distances of the laser beams from one another on the eye to be treated may be modifiable. By way of example, this may be achieved by altering the tilt angles of the deflection surfaces of the first deflection element 26 and/or second deflection element 27 and/or of the beam splitter surfaces of the first polarizing beam splitter 22 and/or second polarizing beam splitter 24. A further option lies in displacing the first deflection element 26 and/or the second deflection element 27 and/or the polarizing beam splitters 22, 24 relative to one another.

The relative positions of the laser beams in relation to one another on the eye may likewise be modifiable. By way of example, this may be implemented by rotating the first beam multiplier element 20 and/or second beam multiplier element 40 about the z-axis and/or about the x-axis and/or y-axis.

It is conceivable that, in the respective beam multiplier element 20, 40, the intermediate beams or the output beams are simultaneously offset from one another (e.g., by different distances between the first polarizing beam splitter or the second polarizing beam splitter and the first deflection element and/or the second deflection element) and make an angle with respect to one another that is not equal to zero degrees (e.g., by an angle not equal to zero degrees of the deflection elements with respect to one another and/or an angle not equal to ninety degrees of the splitter surfaces of the beam splitters with respect to one another). In this case, the beam multiplier element may for example be arranged at a location between a pupil plane and an intermediate image plane.

The beam splitting device 10 may comprise a lens for the spatial offset of the intermediate beams or output beams to one another.

### LIST OF REFERENCE SIGNS

5 Laser source
10 Beam splitting device
20 First beam multiplier element
22 First polarizing beam splitter in the first beam splitting device
23 Splitter surface of the first polarizing beam splitter in the first beam splitting device
24 Second polarizing beam splitter in the first beam splitting device
25 Splitter surface of the second polarizing beam splitter in the first beam splitting device
26, 27 Deflection element of the first beam splitting device
29 Third polarizing beam splitter
40 Second beam multiplier element
42 First polarizing beam splitter in the second beam splitting device
44 Second polarizing beam splitter in the second beam splitting device
46 Deflection element of the second beam splitting device
50, 51 Retarder
60 Laser input beam
61 First portion of the input beam
62 Second portion of the input beam
75, 76, 77 Intermediate beam
80, 81 First component beams
85, 86 Second component beams
90-93 Laser output beam
95 50:50 beam splitter

1.-23. (canceled)

24. A beam splitting device that creates a plurality of laser output beams from a laser input beam,
wherein the beam splitting device comprises a first beam multiplier that creates two intermediate beams from the laser input beam;
wherein the first beam multiplier comprises a first polarizing beam splitter, a second polarizing beam splitter, and at least one first deflection element that deflects an intermediate beam through a selected first angle,
wherein the beam splitting device is configured such that,
a laser input beam that is emitted to the first polarizing beam splitter in the first beam multiplier element is split into a first intermediate beam and a second intermediate beam by operation of the first polarizing beam splitter in the first beam multiplier element, with two intermediate beams spanning an x-y-plane,
the second intermediate beam is deflected by the first deflection element through a selected first angle, and
the first intermediate beam and the second intermediate beam are emitted to the second polarizing beam splitter in the first beam multiplier element, such that the first intermediate beam and the second intermediate beam are emitted from the second polarizing beam splitter in the first beam multiplier element in a manner offset in parallel to one another or with a first angular difference.

25. The beam splitting device as claimed in claim 24, wherein the selected first angle approximates 90° or 180°.

26. The beam splitting device as claimed in claim 24, wherein the first angular difference is selected from a group consisting of less than 3 mrad, less than 1.4 mrad, and less than 0.6 mrad.

27. The beam splitting device as claimed in claim 24, further comprising:
a second beam multiplier having a third polarizing beam splitter, a second deflection element, and a fourth polarizing beam splitter, wherein the second beam multiplier is configured such that,
if the two intermediate beams are directed to the third polarizing beam splitter in the second beam multiplier,
the two intermediate beams are each split into a first component beam and a second component beam by operation of the third polarizing beam splitter in the second beam multiplier,
the second component beams are deflected in the second beam multiplier element through a selected second angle, and
the first component beams and the second component beams are directed to the fourth polarizing beam splitter in the second beam multiplier element in such a way that the four laser output beams emerge from the second polarizing beam splitter in the second beam multiplier in a manner offset in parallel to one another or with a second angular difference.

28. The beam splitting device as claimed in claim 25, wherein the selected second angle approximates 90° or 180°.

29. The beam splitting device as claimed in claim 25, wherein the second angular distance is selected from a group consisting of less than 3 mrad, less than 1.4 mrad, and less than 0.6 mrad.

30. The beam splitting device as claimed in claim 24, wherein at least one of the first polarizing beam splitter in the first beam multiplier element and the third polarizing beam splitter in the second beam multiplier element is aligned vis-à-vis the second polarizing beam splitter or the fourth polarizing beam splitter in a manner rotated through a selected third angle, approximating 90°, about an axis, which is referred to as the z-axis and which runs perpendicular to the x-y-plane, a first distance between the first polarizing beam splitter and the first deflection element being greater than or less than a second distance between the second polarizing beam splitter and the first deflection element.

31. The beam splitting device as claimed in claim 24, wherein at least one of the first polarizing beam splitter in at least one of the first beam multiplier and the third polarizing beam splitter in the second beam multiplier is aligned vis-à-vis the second polarizing beam splitter or the fourth polarizing beam splitter in a manner rotated through an angle not equal to 90°, in the range selected from a group consisting of 90°±3 mrad/2, 90°±1.4 mrad/2, and 90°±0.6 mrad/2, about an axis, which is referred to as the z-axis and which runs perpendicular to the x-y-plane.

32. The beam splitting device as claimed in claim 24, wherein at least one of the first beam multiplier and the second beam multiplier comprise a respective third deflection element, with a deflection surface of the first deflection element extending substantially parallel to a deflection surface of the third deflection element, with the distance between the first polarizing beam splitter and the first deflection element being greater than or less than the distance between the second polarizing beam splitter and the third deflection element.

33. The beam splitting device as claimed in claim 24, wherein at least one of the first beam multiplier and the second beam multiplier comprise a respective third deflection element, with a first deflection surface of the first deflection element not extending parallel to a second deflection surface of the third deflection element and being oriented at an angle selected from a group consisting of up to 1.5 mrad, up to 0.7 mrad, and up to 0.3 mrad.

34. The beam splitting device as claimed in claim 24, wherein the first polarizing beam splitter and the second polarizing beam splitter are arranged adjacent to one another.

35. The beam splitting device as claimed in claim 24, wherein the polarizations of the first intermediate beam, the component beam or both and of the second intermediate beam, the component beam or both are each rotated by use of a half-wave plate or two quarter-wave plates before the two intermediate beams or component beams strike the second polarizing beam splitter.

36. The beam splitting device as claimed in claim 25, further comprising a retarder arranged between the first beam multiplier and the second beam multiplier.

37. The beam splitting device as claimed in claim 36, wherein the retarder comprises a half-wave plate or a quarter-wave plate.

38. The beam splitting device as claimed in claim 24, wherein the deflection surface of the first and/or second deflection element in the first beam multiplier and/or of the third and/or fourth deflection element in the second beam multiplier and/or a splitter surface of the second beam splitter in the first beam multiplier and/or of the fourth beam splitter in the second beam multiplier is tilted through a selected third angle with respect to the x-y-plane about an axis parallel to the x-axis, in such a way that the output beams are not in the x-y-plane.

39. The beam splitting device as claimed in claim 24, wherein the beam splitting device is configured such that a distance between the intermediate beams and/or the laser output beams is kept constant in a plane perpendicular to the x-axis.

40. The beam splitting device as claimed claim 24, wherein the beam splitting device is configured such that a distance of the intermediate beams, and/or the laser output beams from one another immediately downstream of the second beam multiplier element is in each case less than 10-times the largest diameter of the laser output beams in a plane substantially perpendicular to the x-axis.

41. The beam splitting device as claimed claim 40, wherein the beam splitting device is configured such that a distance of the intermediate beams and/or the laser output beams from one another immediately downstream of the second beam multiplier element is in each case 1 less than four-times, the largest diameter of the laser output beams in a plane substantially perpendicular to the x-axis.

42. The beam splitting device as claimed in claim 24, wherein the laser output beams are not in a straight line in a plane perpendicular to the x-axis.

43. An ophthalmological laser therapy system for treating a patient's eye, comprising:
a laser generation device for emitting a laser input beam,
a beam splitting device as claimed in claim 24 that creates two or four laser output beams from the laser input beam, and
a scanning device for moving the laser output beams over the patient's eye.

44. The ophthalmological laser therapy system as claimed in claim 43, wherein the laser therapy system is configured such that distances of the laser output beams from one another do not change when moving the laser output beams over the patient's eye.

45. A method for scanning a patient's eye with two or four laser output beams, the method comprising:
radiating a laser input beam into a beam splitting device as claimed in claim 24;
emitting two or four laser output beams from the beam splitting device; and
scanning the laser output beams over at least a portion of the patient's eye.

46. The method as claimed in claim 45, further comprising moving the laser output beams over the patient's eye, in such a way that distances of the laser output beams from one another do not change when moving the laser output beams over the patient's eye.

47. The method as claimed in claim 45, wherein the laser output beams are not in a straight line in a plane extending perpendicular to the direction of the laser input beam.

48. A method for splitting a laser input beam into two laser output beams, the method comprising:
emitting a laser input beam to a first polarizing beam splitter in a first beam multiplier;
splitting the laser input beam into a first intermediate beam and a second intermediate beam by use of the first polarizing beam splitter in the first beam multiplier;
deflecting the second intermediate beam in the first beam multiplier element through a selected angle approximating 90° or approximating 180°; and
directing the first intermediate beam and the second intermediate beam at a second polarizing beam splitter in the first beam multiplier in such a way that the first intermediate beam and the second intermediate beam emerge from the second polarizing beam splitter in the first beam multiplier element parallel to one another or with a selected angular difference, selected from a group consisting of less than 3 mrad, less than 1.4 mrad, and less than 0.6 mrad.

49. The method as claimed in claim 48, further comprising:
emitting the two intermediate beams at a third polarizing beam splitter in a second beam multiplier;
splitting each of the two intermediate beams into component beams including first component beams and second component beams by use of the third polarizing beam splitter in the second beam multiplier;
deflecting the second component beams in the second beam multiplier through a selected angle, approximating 90° or approximating 180°; and
directing the first component beams and the second component beams at a fourth polarizing beam splitter in the second beam multiplier element, in such a way that four laser output beams emerge from the second polarizing beam splitter in the second beam multiplier in a manner offset parallel to one another or with a selected angular difference selected from a group consisting of less than 3 mrad, less than 1.4 mrad, and less than 0.6 mrad.

50. The method as claimed in claim 49, wherein the second beam multiplier is arranged vis-à-vis the first beam multiplier in a manner rotated through a selected angle, approximating 45° or approximating 90°, about an axis, which is referred to as the x-axis and runs parallel to the laser input beam.

51. The method as claimed in claim 45, further comprising arranging a half-wave plate or a quarter-wave plate between the first beam multiplier and the second beam multiplier.
